(19) United States
(12) Patent Application Publication
(10) Pub. No.: US 2016/0175369 A1
(43) Pub. Date: Jun. 23, 2016

(54) COMPOSITIONS AND METHODS FOR REDUCING INFECTION IN POULTRY

(71) Applicant: Nutrition Physiology Company, LLC, Guymon, OK (US)

(72) Inventor: Steven P. Lerner, Snellville, GA (US)

(21) Appl. No.: 14/973,034

(22) Filed: Dec. 17, 2015

Related U.S. Application Data
(60) Provisional application No. 62/093,149, filed on Dec. 17, 2014.

(51) Int. Cl. A61K 35/747; A61K 9/00; A61K 39/012

(52) U.S. Cl. CPC A61K 35/747; A61K 39/012; A61K 9/0017; A61K 9/0056; A23K 1/1826; A23K 1/009; A61K 2039/552

(57) ABSTRACT

Methods and compositions are hereby disclosed for reducing coccidiosis in birds, such as chickens or turkeys, among others. The methods include administering to the bird a lactic acid producing bacterium (LAB) alone or in combination with a vaccine against coccidiosis.

### RELATED APPLICATIONS

This application claims priority to U.S. Patent Application No. 62/093,149, filed Dec. 17, 2014, which is hereby incorporated by reference in its entirety.

### BACKGROUND

I. Field of the Invention

The present disclosure pertains to the use of lactic acid bacteria to reduce pathogen infection in animals, including poultry. More particularly, the disclosure relates to the use of lactic acid bacteria as a supplement to reduce coccidiosis in poultry.

II. Description of Related Art

Reducing pathogen infection is important in the poultry industry. Lactic acid producing bacteria (also referred to as “lactic acid bacteria” or “LAB” in this disclosure) have been shown to help reduce pathogen infection in ruminants. See, e.g., U.S. Pat. No. 7,063,836. The compositions and methods disclosed in U.S. Pat. No. 7,063,836 help reduce the numbers of enteropathogens such as E. coli O157:H7. Because ruminant digestive systems are different from those of birds, and because ruminants and birds have different native microflora, the same LAB supplementation that works in ruminants may not work in poultry.

Various pathogens are known to cause infectious diseases in birds. Because infectious diseases may spread rapidly in a poultry farm, minimizing the risk of infectious diseases is critical. One example of such infectious disease is coccidiosis. Coccidiosis is a parasitic disease of the intestinal tract of animals, including birds. Coccidiosis is typically caused by protozoa of the phylum Apicomplexa, family Eimeriidae. In poultry, most species that cause coccidiosis belong to the genus Eimeria. The infection is characterized by parasite replication in host cells with extensive damage to the intestinal mucosa. Symptoms of coccidiosis may range from decreased growth rate to severe diarrhea and death. Some milder form of coccidiosis may cause secondary infection, for example, infection by Clostridium.

Various methods have been used to prevent coccidiosis. For instance, wire floors have been used to separate birds from droppings. Vaccination and anticoccidial drugs have also been used to control coccidiosis.

### SUMMARY

The present disclosure advances the art by providing compositions and methods for reducing pathogenic infection in poultry. More specifically, the disclosed compositions and methods help reduce incidence of coccidiosis in birds.

In one embodiment, a composition is provided for reducing pathogen infection in a bird, wherein the composition may contain an effective amount of at least one lactic acid producing bacterium (also referred to as “lactic acid bacterium” or “LAB” in this disclosure). In one aspect, an effective amount is an amount of the lactic acid producing bacterium that is capable of reducing Coccidiosis in the bird. In another aspect, an effective amount is an amount of the lactic acid producing bacterium that reduces mortality rate of a group of birds by at least 30%, 40%, 50%, 60%, 70%, or 80% when administered to a group of birds. In another aspect, an effective amount is an amount of the lactic acid producing bacterium that reduces the probability of the bird dying at age 21 days by at least 30%, 40%, 50%, 60%, or 80% when administered to the bird.

In another embodiment, the bird may be a member selected from the taxonomic order Anseriformes, Galliformes, or Columbiformes. Examples of birds may include but are not limited to a chicken, a quail, a grouse, a duck, a goose, a swan, a turkey, a pigeon, a partridge, a pheasant, a fowl, among others.

In one aspect, supplementing LAB to a bird may enhance feed efficiency in the bird. In another aspect, the lactic acid bacteria may reduce infection of the bird by various pathogens, or reduce pathogen contamination of the carcass of the bird. In another aspect, the lactic acid bacteria may help reduce coccidiosis in the bird.

In another embodiment, the effective amount of LAB is an amount of the lactic acid producing bacterium that is capable of reducing coccidiosis but does not interfere with effectiveness of a vaccine against coccidiosis. In another embodiment, the lactic acid bacteria may boost the effect of an anti-coccidiosis vaccine in the bird, such that co-administration of LAB and an anti-coccidiosis vaccine is at least 20%, 30%, 40%, 50%, 60%, 80% more effective in reducing coccidiosis than administration of anti-coccidiosis vaccine alone. In one aspect, the lactic acid bacteria and an anti-coccidiosis vaccine may have synergistic effect in reducing coccidiosis when they are co-administered to a bird.

In one embodiment, the disclosed method may further include a step (b) of administering to the bird a vaccine against coccidiosis, wherein step (a) and step (b) may be carried out at the same time or sequentially. In one aspect, LAB and the vaccine may be pre-mixed and administered at the same time. In another aspect, the LAB and the vaccine may be administered separately but at the same time. In another aspect, the LAB and the vaccine may be administered separately and sequentially, with LAB administration preceding vaccine administration, or vice versa.

In another embodiment, the LAB may be fed to a bird as a dietary supplement to enhance the feed efficiency and/or to reduce pathogenic infection. In one aspect, the LAB may be fed to the bird at a dosage that is sufficient to reduce the amount of at least one pathogen in the digestive system of the bird by at least 20%, 30%, 40%, 50%, 60%, 80% or more, as compared to the amount of said at least one pathogen in an untreated bird. For purpose of this disclosure, the term “at least one pathogen” may include but not limited to one or more of Salmonella typhimurium, Eimeria, Escherichia coli, Salmonella enterica, Staphylococcus aureus, Listeria monocytogenes, Clostridium perfringens, and Campylobacter jejuni.

In one embodiment, the LAB may be administered to the bird at a dosage that is sufficient to reduce lesion in the gut of the bird by at least 20%, 30%, 40%, 50%, 60%, 70% or more when administered to the bird. In another embodiment, the LAB may be administered to the bird at a dosage that is sufficient to reduce E. coli in the digestive system of the bird by at least 10-fold, 50-fold, 100 fold or more when administered to the bird. As used here, a 10-fold reduction refers to a reduction in colony forming unit (CFU) by one log, for example, from 10⁵ CFU to 10⁴ CFU. Similarly, a 100-fold reduction refers to a reduction in colony forming unit (CFU) by two logs, for example, from 10⁵ CFU to 10³ CFU. In another embodiment, the LAB may be administered to the bird at a dosage that is sufficient to reduce Eimeria in the digestive system of the bird by at least 50%, 60%, 80%, 90%, or 99% when administered to the bird. In another embodiment, the LAB may be administered to the bird at a dosage that is sufficient to reduce Salmonella in the digestive system of the bird by at least 50%, 60%, 80%, 90%, or 99% when administered to the bird.

In one embodiment, the disclosed composition may contain one or more lactic acid producing bacteria (LAB). Examples of the LAB may include but are not limited to the genus of Lactobacillus. In one aspect, at least one of the lactic acid producing bacteria may be Lactobacillus acidophillus. Examples of Lactobacillus strains may include but are not limited to LA51, LA45, NP28 (also known as C28) and L411 strains. In one aspect, more than one lactic acid producing bacteria that belong to the same or different species may be used in the supplement. In another aspect, the composition does not contain significant amount of lactic acid utilizing bacteria. In another aspect, the intake of lactic acid utilizing bacteria via supplementation of the disclosed composition, if any, is less than 100 CFU per day. In another aspect, the composition does not contain lactic acid utilizing bacteria. Examples of lactic acid utilizing bacteria include but are not limited to Propionibacterium freudenreichii, among others.

In one embodiment, a method is disclosed for improving feed utilization in a bird wherein a composition comprising a Lactobacillus strain LA51 is administered to the bird at a dosage of from about 1×10³ to about 1×10¹⁰ CFU per day for each bird. In another embodiment, a method is disclosed for reducing pathogenic infection in a bird wherein a composition comprising a Lactobacillus strain LA51 is administered to the bird at a dosage of from about 1×10³ to about 1×10¹⁰ CFU per day for each bird.

The lactic acid producing bacteria may be administered to the bird separately from regular feed and/or drinks. Alternatively, the bacteria may be administered to the bird along with regular feed and/or drinks. In one aspect, the lactic acid producing bacteria may be pre-mixed with feed or water and administered to the bird in the form of a pre-mix. In another aspect, the LAB may be pre-mixed with feed specific for domesticated birds, for example, feed specific for broiler chickens, before being administered to the birds. In another aspect, the LAB may be formulated so that it is in a form that may be sprayed onto birds in a farm, such as inside a structure or at an open space. In another aspect, the LAB may be sprayed onto a bird at a dosage between 1×10⁶ and 1×10⁹ CFU, or between 1×10⁷ and 1×10⁸ CFU for each strain per bird. In another aspect, the disclosed composition may be either sprayed onto the bird or fed to the bird as a supplement (alone or by mixing with feed and/or water), or by combination of spraying and feeding.

Dosage of the lactic acid bacteria supplement may vary from species to species. The dosage may be determined based on factors such as body weight of the bird, stage of growth, or environmental conditions, among others. In one embodiment, one or more strains of lactic acid bacteria may be administered to the bird at a dosage of between 1×10³ and 1×10¹⁰ CFU for each strain per bird per day. In another aspect, the dosage is between 1×10³ and 1×10⁸ CFU for each strain per bird per day. In another aspect, the dosage is between 1×10⁴ and 1×10⁶ CFU for each strain per bird per day. In another aspect, the dosage is between 1×10⁶ and 1×10⁹ CFU for each strain per bird per day. In another aspect, the dosage is between 1×10⁷ and 1×10⁸ CFU for each strain per bird per day. In another aspect, the dosage is about 1×10⁵ CFU for each strain per bird per day. In another aspect, the dosage is about 1×10⁶ CFU for each strain per bird per day. In another aspect, the dosage is between 1×10⁷ and 5x 10⁷ CFU for each strain per bird per day. In one embodiment, this dosage may be achieved by either spraying, feeding to the bird, or by combination of spraying and feeding.

In one embodiment, the methods may include a step (c) wherein all birds, or at least representatives of the birds and their living environment are assessed to determine if the birds are in need of LAB supplementation and/or vaccination against coccidiosis. For instance, the birds and their housing environment may be assessed to determine if they are at risk of developing coccidiosis. In one aspect, step (c) may help predicting the bird's likelihood of developing coccidiosis. In another aspect, step (c) may be conducted before step (a) and step (b). During step (c), at least one of the following parameters may be determined: body weight gain, feed conversion, lesion, pathogen reduction and combination thereof. In one embodiment, the amount of pathogenic Eimeria in the birds or in their living environment may be measured in step (c), which may be used to determine if the birds are in need of LAB supplementation and/or vaccination against coccidiosis. In another embodiment, the incidence of coccidiosis may be measured in step (d), which may be used to determine if the birds are in need of LAB supplementation and/or vaccination against coccidiosis.

Before the disclosed composition is administered to a bird, the feed efficiency of the bird may be measured or predicted in order to determine if the bird is in need of lactic acid bacteria supplements. The term “feed efficiency” (also referred to as “feed conversion”) is defined as the amount of feed by pound consumed for each bird in order for that bird to gain one pound of weight in the case of all birds other than laying birds. In some instances, kilogram may be used in place of pound as the measurement unit for weight. Feed efficiency may be calculated by dividing the feed intake by the weight gain during the same period. Alternatively, the inverse calculation may be used to calculate feed efficiency. Feed efficiency may fluctuate slightly depending on the different energy levels of different diets. For purpose of this disclosure, the calculation of feed efficiency is based on standard diets containing 3,000-3,200 kcal/kg in the starter, and up to about 3,100-3,300 kcal/kg in the finisher diet.

In one embodiment, the LAB supplementation may be started immediately after hatching, namely from Day 0 of life, and may continue daily until at least 10 days, 15 days, 21 days of life or longer.

The duration of the LAB supplementation varies. In one aspect, a broiler's diet may be supplemented with the LAB continuously for 10-30 days, or 20-60 days daily in order to achieve the desired effects. In another aspect, a turkey's diet may be supplemented with LAB continuously for 20-50 days, or 60-140 days daily, or for a period of about 80-120 days daily. The LAB supplement is ideally provided to the bird continuously on a daily basis during the period of supplementation.

In another embodiment, the method may further include a step (d) to assess the effect of supplementation after at least 2 weeks of LAB supplementation performed in step (a) and/or step (b). During step (d), at least one of the following parameters may be determined: body weight gain, feed conversion, lesion, pathogen reduction and combination thereof. In one embodiment, the amount of pathogenic Eimeria in the birds or in their living environment may be measured in step (d), which may be compared with measurement obtained in step (c). In another embodiment, the incidence of coccidiosis may be measured in step (d), which may be compared with measurement obtained in step (c).

In another embodiment, before the disclosed composition is administered to a bird, the health status of the bird may be measured or predicted to determine if the bird is in need of lactic acid bacteria supplements. In one aspect, mean lesion score in the intestine of the bird may be used as an indicator of the health status of a bird. If the measured or predicted intestinal lesion score is relatively high, lactic acid bacteria supplement may be needed. In another aspect, a mean lesion score of 0.5 or above may indicate that lactic acid bacteria supplement is desirable, or in other words, the birds are in need of lactic acid bacteria supplement. In another aspect, the health status of a bird may be predicted based on empirical data obtained on same or similar breed of birds on same or similar feed and grown under same or similar conditions. After a period of supplements, the health status of the bird may be measured to monitor the effects of the supplements on the health status of the bird.

Birds raised on built-up litter may be more susceptible to pathogen infection than birds raises on fresh litter. The disclosed lactic acid bacteria may be particularly effective in reducing pathogen infection in birds raised on built-up litter. In one aspect, no antibiotic is fed to the birds while they are receiving the LAB supplement as disclosed herein.

### BRIEF DESCRIPTION OF THE DRAWINGS

FIG. 1 summarizes with illustration the effects LAB supplement in a bird.

### DETAILED DESCRIPTION

This disclosure provides improved methods and compositions for reducing coccidiosis in birds. In one embodiment, the disclosed compositions help reduce pathogens and enhance weight gain in poultry.

As used herein, the term “pathogen” refers to a microorganism that may be harmful to a host animal, as well as a microorganism that may not be harmful to the host animal but may be harmful to a human who contacts with or consume the host animal or a product prepared from the host animal. By way of example, the most common pathogens in poultry include but are not limited to Salmonella typhimurium, Eimeria, Escherichia coli, Salmonella enterica, Staphylococcus aureus, Listeria monocytogenes, Clostridium perfringens, and Campylobacter jejuni.

Various commercially available products are described or used in this disclosure. It is to be recognized that these products or associated trade names are cited for purpose of illustration only. Certain physical or chemical properties and composition of the products may be modified without departing from the spirit of the present disclosure. One of ordinary skill in the art may appreciate that under certain circumstances, it may be more desirable or more convenient to alter the physical and/or chemical characteristics or composition of one or more of these products in order to achieve the same or similar objectives as taught by this disclosure. It is to be recognized that certain products or organisms may be marketed under different trade names which may in fact be identical to the products or organisms described herein.

It is to be noted that, as used in this specification and the claims, the singular forms “a,” “an,” and “the” include plural referents unless the context clearly dictates otherwise. Thus, for example, reference to “a pathogen” includes reference to a mixture of two or more pathogens, reference to “a lactic acid producing bacterium” includes reference to one or more lactic acid producing bacteria.

The terms “between” and “at least” as used herein are inclusive. For example, a range of “between 5 and 10” means any amount equal to or greater than 5 but equal to or smaller than 10.

For purpose of this disclosure, the term “precede” means one event or step is started before a second event or step is started.

The dosage of the bacterial supplements is defined by “CFU per day,” which refers to the number of colony forming units of the particular bacterial strain that is administered on the days when the bacterial strain is administered.

The terms “untreated” and “unsupplemented” are used interchangeably, and refer to animals (or birds) that are fed identical or similar diet except for the omission of lactic acid producing bacteria from the diet. The term “performance” refers to one or more of the growth parameters, such as weight gain, feed conversion, and feed efficiency.

Administration of the bacterial supplement may be through oral ingestion with or without feed or water or may be mixed with feed and/or water. The bacterial supplement may be prepared as a pre-mix with feed and/or water or it may be mixed on site at the time of administration. In one aspect, the bacterial supplements are administered along with normal feed or water. In another aspect, the bacteria may be prepared in the form of a lyophilized culture before being mixed with water for spraying or blending with the feed and/or water. The final mixture may be in dry or wet form, and may contain additional carriers that are added to the normal feed of the birds. The normal feed may include one or more ingredients such as cereal grains, cereal grain by-products, or other commercial bird or poultry feed products. The lyophilized cultures may also be added to the drinking water of the birds.

Preparation of the bacterial supplement to be mixed with feed or water may be performed as described in U.S. Pat. No. 7,063, 836. Detection and enumeration of pathogenic bacteria may be conducted as described in Stephens et al. (2007). The contents of these references are hereby expressly incorporated by reference into this disclosure.

In one embodiment, the lactic acid producing bacterium may include one or more of the following: Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, and combinations thereof.

In one embodiment, the lactic acid producing bacterium is Lactobacillus acidophilus or Lactobacillus animalis. Examples of the lactic acid producing bacterium strains may include but are not limited to the LA51, LA45, C28 (NP28), and L411. In another embodiment, the lactic acid producing bacterium strain is LA51. The term Lactobacillus acidophilus/animalis may be used to indicate that either Lactobacillus acidophilus or Lactobacillus animalis may be used. It is worth noting that when strain LA51 was first isolated, it was identified as a Lactobacillus acidophilus by using an identification method based on positive or negative reactions to an array of growth substrates and other compounds (e.g., API 50-CHL or Biolog test). Using modern genetic methods, however, strain LA51 has recently been identified as belonging to the species Lactobacillus animalis (unpublished results). Regardless of the possible taxonomic changes for LA51, the strain LA51 remains the same as the one that has been deposited with ATCC.

Lactobacillus strains C28, LA45 and LA51 strains were deposited with the American Type Culture Collection (ATCC) on May 25, 2005 and have the Deposit numbers of PTA-6748, PTA-6749 and PTA-6750, respectively. Lactobacillus strain L411 was deposited with the American Type Culture Collection (ATCC) on Jun. 30, 2005 and has the Deposit number PTA-6820. These deposits were made in compliance with the Budapest

Treaty requirements that the duration of the deposit should be for thirty (30) years from the date of deposit or for five (5) years after the last request for the deposit at the depository, or for the enforceable life of a patent that results from this application, whichever is longer. The strains will be replenished should it become non-viable at the Depository.

The following examples are provided to illustrate the present disclosure, but are not intended to be limiting. The feed ingredients and supplements are presented as typical components, and various substitutions or modifications may be made in view of this disclosure by one of skills in the art without departing from the principle and spirit of the present invention.

Certain feeding tests described in the Examples contain ingredients that are in a size suitable for a small-scale setting. It is important to note that these small scale tests may be scaled up and the principle of operation and the proportion of each ingredient in the system may equally apply to a larger-scale feeding system. Several animal trials are described in the Examples. Unless otherwise specified, multiple duplicates were included in each trial and all data presented are means calculated from the duplicates and the results are statistically significant with P smaller than 0.05. Unless otherwise specified, the percentages of ingredients used in this disclosure are on a w/w basis.

### EXAMPLE 1

### LAB Supplement Improves Feed Efficiency and Intestinal Lesions Under Mild Challenge Conditions in Chickens

This example describes the effect of LAB on feed efficiency and vaccination against coccidiosis when supplemented to newly hatched chickens as compared to vaccine, LAB plus vaccine and untreated controls. The LAB used in this Example is LA51 (aka NP51).

The test period began on the day of hatch of the chicks (Trial Day 0). The chicks were fed a commercial-type feed until the end of the study. On Trial Day 0 (or day of hatching), a total of 640 chicks were randomly assigned into 4 groups: (1) untreated control, (2) a commercially available vaccine against coccidiosis in chicken (“vaccine-C” in this Example), (3) LAB, (4) vaccine-C and LAB.

All birds designated to receive LAB in feed were sprayed with LAB at day of age (>5×10⁷ cfu/bird). LAB was also added to the corn-soy based diet feed to achieve a dosage between 1 ×10⁷ and 5×10⁷ cfu per bird per day.

Built up litter bedding (with litter crust from previous trial) from at least three previous grow-outs was used to place minimum, but normal, “field condition stress” on the birds. For challenge, an additional bacteria challenge was added to the built up litter of all groups on the 7^{th} and 10^{th} day post-hatch: C. perfringens (10⁴ cfu per bird, considered to be a MILD stress level), E. acervulina (10⁴ cfu per bird), and E. coli (10⁶ cfu per bird).

At the completion of the test period (21 days of age), weight, feed conversion, gut lesion scores (at necropsy), and mortality were measured. Statistical evaluation was based on individual birds (10 replicates per group). Lesion scoring was done in the following manner: Scores are 0-4:0=No lesions found; 1=Slight redness but no cell sluffing (mucus); 2=Moderate redness and/or slight cell sluffing; 3=Severe redness and/or severe cell sluffing; 4=Actual bleeding observed. All data presented in this Example are mean values based on at least 10 replicates per group.

Table 1 shows body weight gain of Broiler Chicks after 21 days of supplementation.

TABLE 1
Body Weight Gain

| | Control | Vaccine-C | LAB | LAB and Vaccine-C |
|---|---|---|---|---|
| Body Weight (lb) | 1.873 | 1.880 | 1.898 | 1.912 |

Table 2 shows the Feed Conversion of Broiler Chicks after 21 days of supplementation.

TABLE 2
Feed Conversion

| | Control | Vaccine-C | LAB | LAB and Vaccine-C |
|---|---|---|---|---|
| Feed Conversion | 1.292 | 1.284 | 1.275 | 1.262 |

Table 3 shows the Lesion Scores of Broiler Chicks after 21 days of supplementation.

TABLE 3
Lesion Scores

| | Control | Vaccine-C | LAB | LAB and Vaccine-C |
|---|---|---|---|---|
| Lesion Scores | 0.441 | 0.417 | 0.334 | 0.264 |

### EXAMPLE 2

### LAB Supplement Improves Feed Efficiency and Intestinal Lesions Under Severe Challenge with Eimeria and E. coli in Chickens

This example describes the effect of LAB on feed efficiency and coccidiosis when supplemented to newly hatched chickens as compared to no-supplement controls. The LAB used in this Example is LA51 (aka NP51).

The test period began on the day of hatch of the chicks (Trial Day 0). The chicks were fed a commercial-type feed until the end of the study. On Trial Day 0 (or day of hatching), a total of 320 chicks were randomly assigned into 2 groups: (1) untreated control, and (2) LAB.

All birds designated to receive LAB in feed were sprayed with LAB at day of age (>5×10⁷ cfu/bird). LAB was also added to the corn-soy based diet feed to achieve a dosage between 1 ×10⁷ and 5×10⁷ cfu per bird per day.

Built up litter bedding (with litter crust from previous trial) from at least three previous grow-outs was used to place minimum, but normal, “field condition stress” on the birds. For challenge, an additional bacteria challenge was added to the built up litter of all groups on the 7^{th} and 10^{th} day post-hatch: C. perfringens (10⁴ cfu per bird, considered to be a MILD stress level), E. acervulina (10⁵ cfu per bird), E. maxima (10⁵ cfu per bird), and E. coli (10⁶ cfu per bird).

At the completion of the test period (21 days of age), weight, feed conversion, gut lesion scores (at necropsy), and mortality were measured. Statistical evaluation was based on individual birds (10 replicates per group). Lesion scoring was done in the following manner: Scores are 0-4:0=No lesions found; 1=Slight redness but no cell sluffing (mucus); 2=Moderate redness and/or slight cell sluffing; 3=Severe redness and/or severe cell sluffing; 4=Actual bleeding observed. All data presented in this Example are mean values based on at least 10 replicates per group.

Table 4 shows body weight gain of Broiler Chicks after 21 days of supplementation. LA51 supplement increased body weight gain by about 6.2%.

TABLE 4
Body Weight Gain

| | Control | LAB |
|---|---|---|
| Body Weight (lb) | 1.595 | 1.693 |

Table 5 shows the feed conversion of Broiler Chicks after 21 days of supplementation. LA51 supplement reduced feed conversion by about 3.1%.

TABLE 5
Feed Conversion

| | Control | LAB |
|---|---|---|
| Feed Conversion | 1.391 | 1.348 |

Table 6 shows the lesion scores of Broiler Chicks after 21 days of supplementation. LA51 supplement reduced lesion scores by about 72%.

TABLE 6
Lesion Scores

| | Control | LAB |
|---|---|---|
| Lesion Scores | 1.149 | 0.324 |

Table 7 shows the mortality rate of Broiler Chicks after 21 days of supplementation. LA51 supplement reduced mortality by about 4.4 percentage point.

TABLE 7
Mortality Rate

| | Control | LAB |
|---|---|---|
| Percent Mortality | 8.13 | 3.75 |

### EXAMPLE 3

### LAB Supplement Improves Feed Efficiency and Intestinal Lesions Under Severe Challenge with Eimeria and Clostridium in Chickens

This example describes the effect of LAB on feed efficiency and coccidiosis when LAB were supplemented to newly hatched chickens as compared to no-supplement controls. The LAB used in this Example is LA51 (aka NP51).

The test period began on the day of hatch of the chicks (Trial Day 0). The chicks were fed a commercial-type feed until the end of the study. On Trial Day 0 (or day of hatching), a total of 320 chicks were randomly assigned into 2 groups: (1) untreated control, and (2) LAB.

All birds designated to receive LAB in feed were sprayed with LAB at day of age (>5×10⁷ cfu/bird). LAB was also added to the corn-soy based diet feed to achieve a dosage between 1 ×10⁷ and 5×10⁷ cfu per bird per day.

Built up litter bedding (with litter crust from previous trial) from at least three previous grow-outs was used to place minimum, but normal, “field condition stress” on the birds. For challenge, an additional bacteria challenge was added to the built up litter of all groups on the 7^{th} and 10^{th} day post-hatch: C. perfringens (10⁸ cfu per bird, considered to be a severe stress level), E. acervulina (10⁴ cfu per bird), and E. maxima (10³ cfu per bird).

At the completion of the test period (21 days of age), weight, feed conversion, gut lesion scores (at necropsy), and mortality were measured. Statistical evaluation was based on individual birds (10 replicates per group). Lesion scoring was done in the following manner: Scores are 0-4:0=No lesions found; 1=Slight redness but no cell sluffing (mucus); 2=Moderate redness and/or slight cell sluffing; 3=Severe redness and/or severe cell sluffing; 4=Actual bleeding observed. All data presented in this Example are mean values based on at least 10 replicates per group.

Table 8 shows body weight gain of Broiler Chicks after 21 days of supplementation. LA51 supplement increased body weight gain by about 6.2%.

TABLE 8
Body Weight Gain

| | Control | LAB |
|---|---|---|
| Body Weight (lb) | 1.77 | 1.88 |

Table 9 shows the feed conversion of Broiler Chicks after 21 days of supplementation. LA51 supplement reduced feed conversion by about 4.5%.

TABLE 9
Feed Conversion

| | Control | LAB |
|---|---|---|
| Feed Conversion | 1.388 | 1.326 |

Table 10 shows the lesion scores of Broiler Chicks after 21 days of supplementation. LA51 supplement reduced lesion scores by about 49%.

TABLE 10
Lesion Scores

| | Control | LAB |
|---|---|---|
| Lesion Scores | 1.227 | 0.625 |

Table 11 shows the mortality rate of Broiler Chicks after 21 days of supplementation. LA51 supplement reduced mortality by about 6.3 percentage point.

TABLE 11
Mortality Rate

| | Control | LAB |
|---|---|---|
| Percent Mortality | 9.38 | 3.13 |

### EXAMPLE 4

### LAB Supplement Reduces Gut Pathogen Counts in Chickens

This example describes the effect of LAB on reducing various pathogens in the digestive system of chickens. The LAB used in this Example is LA51 (aka NP51).

The test period began on the day of hatch of the chicks (Trial Day 0). The chicks were fed a commercial-type feed until the end of the study. On Trial Day 0 (or day of hatching), a total of 84 chicks were randomly assigned into 2 groups: (1) untreated control, and (2) LAB.

Each treatment was administered daily throughout the test period (hatch to 21 days of age). Test feed was mixed fresh daily. Necropsy and Measurements: On day 21 days of age and following live body weight measurements, gross necropsy was conducted on 40 birds per treatment, small intestine lesion scoring determined and then crop and small intestine gut contents collected. These contents were sealed and placed immediately in an ice bath (see procedures below. Statistical evaluation was based on individual birds (40 replicates per treatment. For all birds designated to receive LAB, LAB was added to the corn-soy based diet feed to achieve a dosage between 1 ×10⁷ and 5×10⁷ cfu per bird per day.

All test birds were kept on test feed throughout the 21-day test period, including while birds were administered challenge material. At 21 days of age, all birds were sacrificed. Gross Necropsy was conducted and Small Intestine Lesion Scores determined. Crop and Small Intestine contents were collected. Gut content (crop and small intestine separately) Salmonella spp., E. coli, and total bacteria (Total Plate Count or Total Viable Count) count were determined.

Fresh built up litter bedding was utilized from at least three previous grow-outs in order to place minimum but normal “field condition stress” on the birds. For challenge, an additional bacteria challenge was added to the built up litter of all groups on the 7^{th} and 10^{th} day post-hatch: C. perfringens (10⁴ cfu per bird), E. acervulina (10⁴ cfu per bird), Salmonella spp (10⁴ per bird), and E. coli (10⁶ cfu per bird).

On day 21 (21 days of age), chicken gut contents were collected from the following intestine areas: crop and small intestine. Chicken gut contents from each area were placed in separate containers, measured (by weight or grams) and then contents brought up to 100 grams by adding nutrient medium.

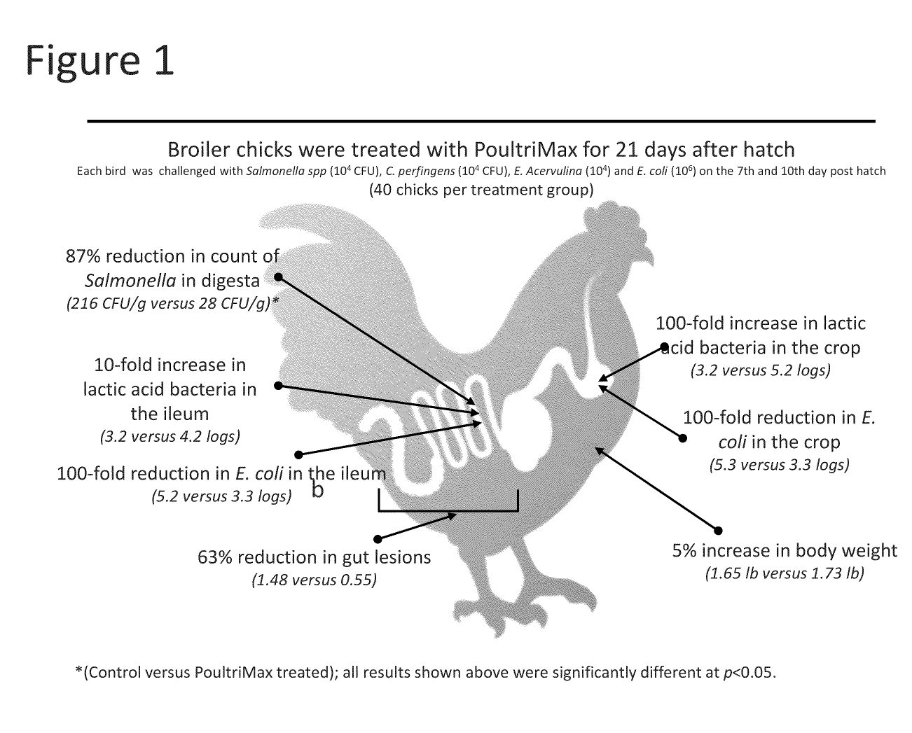

As shown in FIG. 1, after 21 days of daily LAB supplementation, lactic acid bacteria in the crop increased by about 100-fold (5.2 logs vs. 3.2 logs), and by about 10-fold in the ileum (4.2 logs vs. 3.2 logs) as compared to the control group that received no LAB supplementation. LAB supplementation reduced E. coli in the crop by about 100-fold (3.3 logs vs. 5.3 logs), and reduced E. coli in the ileum by about 100-fold (3.3 logs vs. 5.2 logs). LAB supplementation also reduced Salmonella in the digesta by about 87% (28 CFU per gram vs. 216 CFU per gram). A 5% increase in body weight and a 63% reduction gut lesions were also observed in the LAB supplementation group as compared to the control group.

### EXAMPLE 5

### LAB Supplement Improves Feed Efficiency and Reduces Mortality in Turkeys

This example describes the effect of LAB on feed efficiency and vaccination against coccidiosis when supplemented to newly hatched turkeys as compared to vaccine, LAB plus vaccine and untreated controls. The LAB used in this Example is LA51 (aka NP51).

The test period began on the day of hatch of the young turkeys (Trial Day 0). The turkeys were fed a commercial-type feed until the end of the study. On Trial Day 0 (or day of hatching), a total of 600 turkeys were randomly assigned into 4 groups: (1) untreated control, (2) a commercially available vaccine against coccidiosis in turkey (“vaccine-T” in this Example), (3) LAB, (4) vaccine-T and LAB.

All birds designated to receive LAB in feed were sprayed with LAB at day of age (>5×10⁷ cfu/bird). LAB was also added to the corn-soy based diet feed to achieve a dosage between 1 ×10⁷ and 5×10⁷ cfu per bird per day.

New dry litter bedding was used at Day one of age. For challenge, an additional bacteria challenge was added to the litter of all groups on the 7^{th} and 10^{th} day post-hatch: C. perfringens (10⁴ cfu per bird, considered to be a MILD stress level), E. acervulina (10⁴ cfu per bird), and E. coli (10⁶ cfu per bird).

At the completion of the test period (21 days of age), weight, feed conversion, gut lesion scores (at necropsy), and mortality were measured. Statistical evaluation was based on individual birds (10 replicates per group). Lesion scoring was done in the following manner: Scores are 0-4:0=No lesions found; 1=Slight redness but no cell sluffing (mucus); 2=Moderate redness and/or slight cell sluffing; 3=Severe redness and/or severe cell sluffing; 4=Actual bleeding observed. All data presented in this Example are mean values based on at least 10 replicates per group.

Table 12 shows body weight gain of the turkeys after 21 days of supplementation.

TABLE 12
Body Weight Gain

| | Control | Vaccine-T | LAB | LAB and Vaccine-T |
|---|---|---|---|---|
| Body Weight (lb) | 1.900 | 1.914 | 1.942 | 1.957 |

Table 13 shows feed conversion of the turkeys after 21 days of supplementation.

TABLE 13
Feed Conversion

| | Control | Vaccine-T | LAB | LAB and Vaccine-T |
|---|---|---|---|---|
| Feed Conversion | 1.346 | 1.324 | 1.291 | 1.275 |

Table 14 shows mortality rate of the turkeys after 21 days of supplementation.

TABLE 14
Mortality

| | Control | Vaccine-T | LAB | LAB and Vaccine-T |
|---|---|---|---|---|
| % Mortality | 7.53 | 5.33 | 2 | 2.67 |

### EXAMPLE 6

### Long-Term Large-Scale Test of LAB Supplement in Turkeys

This example describes the effect of LAB on pathogen reduction and mortality when supplemented to newly hatched turkeys for an extended period of time as compared to turkeys fed with a commercial supplement. The LAB used in this Example is LA51 (aka NP51).

The LA51 treated group was divided into two subgroups. For one subgroup, LA51 was added to their diet to provide the correct dosage per bird. For the other subgroup, LA51 was sprayed onto each poult at day of age in the commercial hatchery and LA51 added to their diet to provide the correct dosage per bird per day.

Body weight, feed conversion, lesion scores and mortality data were collected as described in Example 5.

At the end of the 17-week trial, as compared the commercial product, LA51 supplemented group shows 44% reduction in gut lesions (Score of 0.6 vs. 1.1 in the commercial product group). The group supplemented with LA51 also gained 3.5% more weight as compared to the group having the commercial product (23.6 lb vs. 22.8 lb in the commercial product group).

Table 15 shows the mortality of the LA51 supplemented turkeys as compared to control group supplemented with the commercial product.

TABLE 15
Mortality

| | LAB | LAB in | Commercial |
|---|---|---|---|
| Mortality at | Spray/feed | feed only | product |
| 2 weeks | 1.3% | 3.5% | 4.0% |
| 6 weeks | 1.4% | 4.0% | 6.0% |
| 11 weeks | 2.2% | 6.6% | NA |
| 15 weeks | 3.1% | 8.0% | 9.3% |
| 17 weeks | 3.5% | 9.6% | 12.6% |

We claim:

1. A composition for reducing pathogen infection in a bird, said composition comprising an effective amount of a lactic acid producing bacterium, said effective amount being an amount of said lactic acid producing bacterium that is capable of reducing coccidiosis in said bird.

2. The composition of claim 1, wherein said composition is in a form that can be sprayed.

3. The composition of claim 1, wherein said effective amount is an amount of the lactic acid producing bacterium that reduces lesion in the gut of said bird by at least 30% when administered to said bird.

4. The composition of claim 1, wherein said effective amount is an amount of the lactic acid producing bacterium that reduces E. coli in the gut of said bird by at least 10-fold when administered to said bird.

5. The composition of claim 1, wherein said effective amount is an amount of the lactic acid producing bacterium that reduces Salmonella in the gut of said bird by at least 60% when administered to said bird.

6. The composition of claim 1, wherein said effective amount is an amount of the lactic acid producing bacterium that reduces mortality rate of a group of birds by at least 50% when administered to said group of birds.

7. The composition of claim 1, wherein said lactic acid producing bacterium is a strain selected from the group consisting of LA51, LA45, NP28, and L411 strains.

8. The composition of claim 1, further comprising a vaccine against Coccidiosis.

9. The composition of claim 1, wherein said effective amount is an amount of the lactic acid producing bacterium that is capable of reducing Coccidiosis without interfering with effectiveness of a vaccine against Coccidiosis.

10. The composition of claim 1, wherein said effective amount is an amount of the lactic acid producing bacterium that enhances effectiveness of a vaccine against Coccidiosis by at least 20%.

11. The composition of claim 1, wherein said lactic acid producing bacterium is supplemented to said bird at a dosage of between 1×10³ and 1×10¹⁰ CFU per day for each bird.

12. A method for reducing pathogen infection in a bird, said method comprising:
(a) supplement to said bird a composition comprising an effective amount of a lactic acid producing bacterium, said effective amount being an amount of said lactic acid producing bacterium that is capable of reducing Coccidiosis in said bird.

13. The method of claim 12, wherein said lactic acid producing bacterium is pre-mixed with feed or water for said bird before being supplemented to said bird.

14. The method of claim 12, wherein said lactic acid producing bacterium is sprayed onto said bird.

15. The method of claim 12, wherein said effective amount is an amount of the lactic acid producing bacterium that reduces lesion in the gut of said bird by at least 30% when administered to said bird.

16. The method of claim 12, wherein said lactic acid producing bacterium being supplemented to the bird at a dosage range of between 1×10³ and 1×10¹⁰ CFU of lactic acid producing bacterium per day for each bird.

17. The method of claim 12, wherein said composition comprises a lactic acid producing bacterium and a vaccine against coccidiosis.

18. The method of claim 12, further comprising
(b) administering to said bird a vaccine against coccidiosis,
wherein said step (a) and step (b) are carried out at the same time or sequentially.

19. The method of claim 12, wherein said pathogen is selected from the group consisting of Eimeria, Escherichia coli, Salmonella enterica, Staphylococcus aureus, Listeria monocytogenes, Clostridium perfringens, and Campylobacter jejuni.

20. The method of claim 12, wherein said bird is from the taxonomic order Anseriformes, Galliformes, or Columbiformes.

21. The method of claim 12, wherein said bird is selected from the group consisting of a chicken, a quail, a grouse, a duck, a goose, a swan, a turkey, a pigeon, a partridge, a pheasant, and a fowl.

22. The method of claim 12, wherein said composition does not contain significant amount of lactic acid utilizing bacterium.

23. The method of claim 12, further comprising a step (c) of predicting the likelihood of developing coccidiosis in said bird, said step (c) preceding said step (a).

24. The method of claim 12, further comprising a step (d) of measuring at least one parameter of said bird to assess the effect of the supplement, said step (a) preceding said step (d), said at least one parameter being selected from the group consisting of body weight gain, feed conversion, lesion, pathogen reduction and combination thereof.

25. The method claim 12, wherein said step (a) is performed at least daily starting at Day 0 after hatching until at least about 15 days after hatching.

26. The method of claim 12, wherein said bird is raised on built-up litter.

27. The method of claim 12, wherein the lactic acid producing bacterium is a strain selected from the group consisting of LA51, LA45, NP28, and L411 strains.
